SEQUENCE LISTING (5 sequences)

SEQ ID NO: 1 - protein, 11 aa, Artificial Sequence
  synthetic construct
GGLVPRGSGGC

SEQ ID NO: 2 - protein, 5 aa, Artificial Sequence
  synthetic construct
PRSFL

SEQ ID NO: 3 - protein, 6 aa, Artificial Sequence
  synthetic construct
DPRSFL

SEQ ID NO: 4 - protein, 5 aa, Artificial Sequence
  synthetic construct
PRSFL

SEQ ID NO: 5 - protein, 6 aa, Artificial Sequence
  synthetic construct
LVPRGS

(19) United States
(12) Patent Application Publication
(10) Pub. No.: US 2018/0214476 A1
(43) Pub. Date: Aug. 2, 2018

(54) THROMBIN-RESPONSIVE HYDROGELS AND DEVICES FOR AUTO-ANTICOAGULANT REGULATION

(71) Applicants: North Carolina State University, Raleigh, NC (US); The University of North Carolina at Chapel Hill, Chapel Hill, NC (US)

(72) Inventors: Zhen Gu, Apex, NC (US); Jicheng Yu, Raleigh, NC (US); Yuqi Zhang, Raleigh, NC (US); Caterina Gallippi, Cary, NC (US)

(21) Appl. No.: 15/886,152

(22) Filed: Feb. 1, 2018

Related U.S. Application Data
(60) Provisional application No. 62/453,162, filed on Feb. 1, 2017.

(51) Int. Cl. A61K 31/727; A61K 9/00; A61K 9/70; A61K 47/61; A61K 47/65; A61K 47/69

(52) U.S. Cl. CPC A61K 31/727; A61K 9/0021; A61K 47/6903; A61K 47/61; A61K 47/65; A61K 9/703

(57) ABSTRACT

The present disclosure relates to a thrombin-responsive closed-loop patch for prolonged heparin delivery in a feedback-controlled manner. The microneedle-based patch can sense the activated thrombin and subsequently release heparin to prevent coagulation in the blood flow. The patch can be transcutaneously inserted into skin without drug leaking and can sustainably regulate blood coagulation in response to thrombin.

### CROSS REFERENCE TO RELATED APPLICATIONS

This application claims the benefit of U.S. Provisional Patent Application Ser. No. 62/453,162, filed Feb. 1, 2017, the disclosure of which is expressly incorporated herein by reference.

### STATEMENT REGARDING FEDERALLY SPONSORED RESEARCH

This invention was made with government support under grant number 1160483 awarded by the National Science Foundation. The government has certain rights in the invention.

### FIELD

The present disclosure relates to compounds, compositions, devices, and methods for auto-anticoagulation regulation. Further disclosed are methods for treating or preventing thrombosis.

### BACKGROUND

Thrombosis, a pathological hemostatic condition, has become one of the leading causes of cardiovascular mortalities and morbidities worldwide. The unwanted intravascular blood thrombi can cause vascular occlusions, organ damage, and severe cardiovascular diseases, including myocardial infarction and stroke. As a first line of defense, anticoagulant drugs can prevent and delay the obstruction in blood flow. Heparin (HP), a common anticoagulant, is routinely administered to counteract coagulation activation. Dosing schemes for HP usually involve daily intravenous administration for weeks to months. Unfortunately, systemic (intravenous) or local (catheter) delivery of anticoagulants remains difficult for precise anticoagulant regulation. Under- or over-dosage may lead to dangerous consequences due to either rapid clearance in the body or bleeding complications that may lead to spontaneous hemorrhages. Moreover, it is known that the timely delivery of drugs is critical for cardiovascular patients when an unpredictable attack happens, which makes sustained protection from pathogenesis imperative. Therefore, a controlled and on demand drug delivery system, one that enhances therapeutic efficacy while minimizing side effects and time-to-treatment, is urgently needed for the management of thrombotic diseases.

The compounds, compositions, devices, and methods disclosed herein address these and other needs.

### SUMMARY

Disclosed herein are hydrogels, devices, and methods for auto-anticoagulation regulation. These hydrogels and devices can be used in methods for treating or preventing thrombosis.

In one aspect, disclosed herein is a hydrogel comprising:
a non-peptidic polymer;
a thrombin-cleavable peptide; and
heparin;
wherein the heparin is linked to the non-peptidic polymer by the thrombin-cleavable peptide.

In another aspect, disclosed herein is a device for transport of a material across a biological barrier of a subject comprising:
a plurality of microneedles each having a base end and a tip;
a substrate to which the base ends of the microneedles are attached or integrated; and
a hydrogel, wherein the hydrogel comprises:
   a non-peptidic polymer;
   a thrombin-cleavable peptide; and
   heparin;
   wherein the heparin is linked to the non-peptidic polymer by the thrombin-cleavable peptide.

In a further aspect, disclosed herein is a method for treating or preventing thrombosis in a subject in need thereof, comprising:
providing a microneedle patch to a subject, wherein the microneedle patch comprises:
   a plurality of microneedles each having a base end and a tip;
   a substrate to which the base ends of the microneedles are attached or integrated; and
   a hydrogel, wherein the hydrogel comprises:
      a non-peptidic polymer;
      a thrombin-cleavable peptide; and
      heparin;
      wherein the heparin is linked to the non-peptidic polymer by the thrombin-cleavable peptide;
inserting the microneedles into the biological barrier, wherein the heparin is released from the hydrogel upon cleavage of the thrombin-cleavable peptide.

In one embodiment, the non-peptidic polymer is hyaluronic acid. In one embodiment, the non-peptidic polymer is methacrylated hyaluronic acid.

In one embodiment, the thrombin-cleavable peptide is about 5 to about 30 amino acids in length. In one embodiment, the thrombin-cleavable peptide comprises a sequence of GGLVPRGSGGC (SEQ ID NO:1).

In one embodiment, the heparin is unfractionated heparin.

### BRIEF DESCRIPTION OF THE DRAWINGS

The accompanying figures, which are incorporated in and constitute a part of this specification, illustrate several aspects described below.

FIGS. 1A-1E. Overview of feedback-controlled heparin delivery system. (FIG. 1a) Formation and mechanism of the feedback-controlled heparin delivery system based on thrombin-responsive HAHP (TR-HAHP) conjugate. (FIG. 1b) Schematic of TR-HAHP MN-array patch in response to thrombin. (FIG. 1c) In vitro accumulated FITC labelled HP release from the TR-HAHP hydrogel in several thrombin concentrations at 37° C. (FIG. 1d) Pulsatile release profile of FITC-HP from the TR-HAHP hydrogel (blue: w/o thrombin; pink: w/ thrombin). (FIG. 1e) Fluorescence microscopy images of the TR-HAHP hydrogel in thrombin solution at indicated time points. Scale bar: 1 mm. Error bars indicate s.d. (n=3).

FIGS. 2A-2D. In vitro anticoagulant capacity of the TR-HAHP hydrogel. (FIG. 2a) In vitro analysis of the activated thromboplastin time (aPTT) of untreated (HA), HP treated, non-crosslinked and crosslinked TR-HAHP or NR-HAHP treated plasma. (FIG. 2b) Prothrombin time (PT) tests of plasma incubated with HA, HP, TR-HAHP and NR-HAHP hydrogels. (FIG. 2c) Thrombin clotting time (TCT) of plasma added with various hydrogels. (FIG. 2d) Concentrations of F1+2 fragment after each incubation period (3 h) indicates only the TR-HAHP hydrogel can effectively suppress the thrombin generation during the second incubation. Error bars indicate s.d. (n=3).

FIGS. 3A-3E. Fabrication and in vitro characterization of the TR-HAHP MN-array patch. (FIG. 3a) Photos of MNs array. Scale bar: 1 mm. (FIG. 3b) A fluorescence microscopy image of rhodamine-labelled MN loaded with FITC-labelled TR-HAHP. Scale bar: 200 μm. (FIG. 3c) A SEM image of MNs. Scale bar: 200 μm. (FIG. 3d) Pulsatile release profile of FITC-HP from the TR-HAHP MNs. (blue: w/o thrombin; pink: w/ thrombin). (FIG. 3e) Self-regulated FITC-HP release from MNs in different thrombin solutions. Error bars indicate s.d. (n=3).

FIGS. 4A-4E. In vivo studies of the TR-HAHP patch for thrombosis prevention. (FIG. 4a) Photograph of a mouse transcutaneously administered with the MN-array patch (left). H&E-stained micrograph of mouse skin penetrated by one MN (right top) and the image of the trypan blue staining (right bottom) showing the penetration of the MN patch into the mouse skin. Scale bars are 100 μm and 1 mm, respectively. (FIG. 4b) Kaplan-Meier survival curves for the mice challenged with thrombin injection. Each group was pre-treated with HP i.v. injection or different types of MN patch (HP: 200 U/kg). Shown are eight mice per treatment group. (FIG. 4c) Kaplan-Meier survival curves for thrombotic challenge mouse model 6 h-post MN treatments (HP: 200 U/kg). Shown are eight mice per treatment group. (FIG. 4d) H&E-stained sections of mouse skin tissue at the MN treated sites. Scale bar: 100 μm. (FIG. 4e) Immunofluorescence images of mouse skin tissue stained with TUNEL assay (green) and Hoechst (blue). Scale bar: 50 μm.

FIGS. 5A-5B. LCMS spectra of peptide (FIG. 5a) before and (FIG. 5b) after thrombin cleavage.

FIG. 6. Photographs of TR-HAHP gel before (left) and after (right) UV irradiation.

FIG. 7. Fluoresence microscopy images of the TR-HAHP and NR-HAHP hydrogels in thrombin solutions at indicated time points.

FIG. 8. Release profiles of HP from TR-HAHP and NR-HAHP hydrogels in different concentrations thrombin solutions respectively. Error bars indicate s.d. (n=3).

FIG. 9. Mechanical behavior of one TR-HAHP MN.

FIG. 10. Skin puncture marks at 0 h, 1 h, 2 h and 4 h post-treatment. Scale bar: 2 mm.

FIG. 11. Representative images of FITC-labelled TR-HAHP MNs and NR-HAHP MNs inserted into mice skins after injection of thrombin (1000 U/kg). The white dashed line indicates the boundary of the injected MN. Scale bar: 200 μm.

FIG. 12. Histological observation of the lungs of the mice treated with HA, HP, TR-HAHP and NR-HAHP MNs after challenge of thrombin. Scale bar: 100 μm.

FIG. 13. Histological observation of the lungs of the thrombotic challenge mice 6-h post treatment with HP i.v. injection, HP MNs, and TR-HAHP MNs Scale bar: 100 μm.

FIG. 14. H&E-stained skin sections administered HA, HP, TR-HAHP and NR-HAHP MNs (from left to right) with surrounding tissues 24 h post-administration of the MN-array patch. Scale bar: 100 μm.

FIGS. 15A-15B. Tail transection bleeding time and red blood cell counts after TR-HAHP treatment. (FIG. 15a) Tail transection bleeding time and (FIG. 15b) amounts of red blood cells from the tail wound of animals pretreated with empty HA MN, HP MN, TR-HAHP MN, and NR-HAHP MN. Error bars indicate s.d. (n=5).

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the terms “including,” “containing,” and variations thereof and are open, non-limiting terms. Although the terms “comprising,” “including,” and “containing” have been used herein to describe various embodiments, the terms “consisting essentially of” and “consisting of” can be used in place of “comprising,” “including,” and “containing” to provide for more specific embodiments and are also disclosed.

Disclosed are the components to be used to prepare the disclosed compositions, devices, and patches, as well as the compositions, devices, and patches themselves to be used within the methods disclosed herein. These and other materials are disclosed herein, and it is understood that when combinations, subsets, interactions, groups, etc. of these materials are disclosed that while specific reference of each various individual and collective combination and permutation of these compounds may not be explicitly disclosed, each is specifically contemplated and described herein. For example, if a particular composition or device is disclosed and discussed and a number of modifications that can be made are discussed, specifically contemplated is each and every combination and permutation and the modifications that are possible unless specifically indicated to the contrary. Thus, if a class of components A, B, and C are disclosed as well as a class of components D, E, and F and an example of a combination, or, for example, a combination comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions, devices, and patches. Thus, if there are a variety of additional steps that can be performed it is understood that each of these additional steps can be performed with any specific embodiment or combination of embodiments of the disclosed methods.

It is understood that the components, compositions, devices, and patches disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

“Activities” of a protein, including those relating to “bioactivity,” include, for example, transcription, translation, intracellular translocation, secretion, phosphorylation by kinases, cleavage by proteases, and/or homophilic and heterophilic binding to other proteins.

The term “administering” refers to an administration to a subject that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. Administering can be performed using transdermal microneedle-array patches. The term “parenteral” includes subcutaneous, intravenous, intramuscular, intra-articular, intra-synovial, intrasternal, intrathecal, intrahepatic, intralesional, and intracranial injections or infusion techniques.

“Biocompatible” generally refers to a material and any metabolites or degradation products thereof that are generally non-toxic to the recipient and do not cause any significant adverse effects to the subject.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.”

As used herein, “conjugated” refers to a non-reversible binding interaction.

A “linker” as used herein refers to a molecule that joins adjacent molecules. Generally, a linker has no specific biological activity other than to join the adjacent molecules or to preserve some minimum distance or other spatial relationship between them. In some cases, the linker can be selected to influence or stabilize some property of the adjacent molecules, such as the folding, net charge, or hydrophobicity of the molecule.

The terms “peptide,” “protein,” and “polypeptide” are used interchangeably to refer to a natural or synthetic molecule comprising two or more amino acids linked by the carboxyl group of one amino acid to the alpha amino group of another.

The term “carrier” or “pharmaceutically acceptable carrier” means a carrier or excipient that is useful in preparing a pharmaceutical or therapeutic composition that is generally safe and non-toxic, and includes a carrier that is acceptable for veterinary and/or human pharmaceutical or therapeutic use. As used herein, the terms “carrier” or “pharmaceutically acceptable carrier” can include phosphate buffered saline solution, water, emulsions (such as an oil/water or water/oil emulsion) and/or various types of wetting agents. As used herein, the term “carrier” encompasses any excipient, diluent, filler, salt, buffer, stabilizer, solubilizer, lipid, stabilizer, or other material well known in the art for use in pharmaceutical formulations and as described further below.

As used herein, the term “polymer” refers to a relatively high molecular weight organic compound, natural or synthetic, whose structure can be represented by a repeated small unit, the monomer (e.g., polyethylene, rubber, cellulose). Synthetic polymers are typically formed by addition or condensation polymerization of monomers.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be administered or applied preventively, prophylactically, pallatively or remedially. Prophylactic administration can occur for several days to years prior to the manifestation of symptoms of an infection.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

Disclosed herein are hydrogels, devices, and methods for auto-anticoagulation regulation. These hydrogels and devices can be used in methods for treating or preventing thrombosis.

In one aspect, disclosed herein is a hydrogel comprising:
a non-peptidic polymer;
a thrombin-cleavable peptide; and
heparin;
wherein the heparin is linked to the non-peptidic polymer by the thrombin-cleavable peptide.

In another aspect, disclosed herein is a device for transport of a material across a biological barrier of a subject comprising:
a plurality of microneedles each having a base end and a tip;
a substrate to which the base ends of the microneedles are attached or integrated; and
a hydrogel, wherein the hydrogel comprises:
   a non-peptidic polymer;
   a thrombin-cleavable peptide; and
   heparin;
   wherein the heparin is linked to the non-peptidic polymer by the thrombin-cleavable peptide.

As used herein, the term “non-peptidic polymer” refers to a polymer that does not comprise amino acid oligomers within its polymer backbone. The non-peptidic polymer can be selected from the group consisting of hyaluronic acid, poly(ethylene glycol), poly(propylene glycol), ethylene glycol-propylene glycol copolymer, polyoxyethylated polyol, polyvinyl alcohol, polysaccharide, dextran, polyvinyl ethyl ether, poly(lactic-glycolic acid), biodegradable polymer, lipid polymer, chitin, alginate, and a combination thereof. Derivatives of the above known in the art may be used for the same purpose. In one embodiment, the non-peptidic polymer is hyaluronic acid. In one embodiment, the non-peptidic polymer is methacrylated hyaluronic acid.

In one embodiment, the thrombin-cleavable peptide comprises a sequence of GGLVPRGSGGC (SEQ ID NO:1). In some embodiments, the thrombin-cleavable peptide is about 5 to about 30 amino acids in length. In some embodiments, the thrombin-cleavable peptide can be selected from: GGLVPRGSGGC (SEQ ID NO:1), PRSFL (SEQ ID NO: 2), DPRSFL (SEQ ID NO: 3), or LVPRGS (SEQ ID NO: 4). Thrombin typically cleaves the amide bond at the carboxy-terminus of the arginine residue because the bond structurally resembles the thrombin-cleaved amide linkage in fibrinogen. While examples of thrombin-cleavable peptides have been highlighted above, any sequence that can be cleaved by thrombin may also be used herein.

In some embodiment, the thrombin-cleavable peptide comprises a sequence at least 50% (for example, at least 50%, at least 60%, at least 70%, at least 80%, at least 90%) identical to GGLVPRGSGGC (SEQ ID NO:1). In some embodiment, the thrombin-cleavable peptide comprises GGLVPRGSGGC (SEQ ID NO:1), or a fragment thereof. In some embodiment, the thrombin-cleavable peptide comprises a sequence at least 50% (for example, at least 50%, at least 60%, at least 70%, at least 80%, at least 90%) identical to GGLVPRGSGGC (SEQ ID NO:1), or a fragment thereof.

As used herein, the term “heparin” is generic to both traditional, naturally occurring forms of heparin, as well as heparin derivatives and/or artificial forms of heparin. Heparin is a glycosaminoglycan and acts as an anticoagulant. The molecule has a negative charge density. Some forms of heparin have average molecular weights from about 1 kDa to about 30 kDa, such as between about 12 kDa and about 18 kDa, or about 15 kDa. The term heparin refers to all forms of heparin, including, but not limited to, unfractionated heparin, heparinoids, dermatans, chondroitins, low molecular weight heparin (e.g., tinzaparin (including tinzaparin sodium)), very low molecular weight heparin, and ultra low molecular weight heparin. In some embodiments, the heparin is unfractionated heparin, such as heparin sodium (e.g., heparin sodium USP). The term “low molecular weight heparin” generally refers to heparin in which at least 80% (by weight) of the heparin has a molecular weight of between about 3000 and about 9000 daltons. Non-limiting examples of low molecular weight heparin include tinzaparin, enoxaparin, and dalteparin. The term “very low molecular weight heparin” generally refers to heparin in which at least 80% (by weight) of the heparin has a molecular weight of between about 1500 and about 5000 daltons. Non-limiting examples of very low molecular weight heparin include bemiparin. The term “ultra low molecular weight heparin” generally refers to heparin in which at least 80% (by weight) of the heparin has a molecular weight of between about 1000 and about 2000 daltons. Non-limiting examples of ultra low molecular weight heparin include fondaparinux and semuloparin. Unfractionated heparin (UFH) is a heterogeneous mixture of linear polysaccharide chains with variable molecular weight and biological activity, a well-defined pentasaccharide being its minimal active fragment. Low-molecular weight heparin fractions (LMWH) were developed in the late 1970s and early 1980s by fractionation of the crude UFH, a large proportion of the heparin chains being ineffective as cofactors for antithrombin III, the main inhibitor of thrombin-induced conversion of fibrinogen to fibrin. In some embodiments, the heparin is unfractionated heparin. In some embodiments, the heparin is heparin sodium salt from porcine intestinal mucosa (commercially available, for example, from Sigma-Aldrich (CAT# H3149)).

The microneedles disclosed herein should have the mechanical strength to remain intact while being inserted into the biological barrier, while remaining in place for up to a number of days, and while being removed. In some embodiments, the microneedle must remain intact at least long enough for the microneedle to serve its intended purpose.

The microneedles can have straight or tapered shafts. In one embodiment, the diameter of the microneedle is greatest at the base end of the microneedle and tapers to a point at the end distal the base. The microneedle can also be fabricated to have a shaft that includes both a straight (untapered) portion and a tapered portion. The needles may also not have a tapered end at all, i.e. they may simply be cylinders with blunt or flat tips.

The microneedles can be oriented perpendicular or at an angle to the substrate. In one embodiment, the microneedles are oriented perpendicular to the substrate so that a larger density of microneedles per unit area of substrate can be provided. An array of microneedles can include a mixture of microneedle orientations, heights, or other parameters.

The microneedles can be formed with shafts that have a circular cross-section in the perpendicular, or the cross-section can be non-circular. For example, the cross-section of the microneedle can be polygonal (e.g. star-shaped, square, triangular), oblong, or another shape. The cross-sectional dimensions can be between about 1 μm and 1000 μm, such that the base can be about 100-500 μm, and the tip can be between 1 and 20 μm. In one embodiment, the microneedle can be approximately 300 μm at the base, and approximately 5 μm at the tip.

The length of the microneedles typically is between about 10 μm and 1 mm, preferably between 400 μm and 1 mm. In one embodiment, the length (or height) of the microneedle is about 600 μm. The length is selected for the particular application, accounting for both an inserted and uninserted portion. An array of microneedles can include a mixture of microneedles having, for example, various lengths, outer diameters, inner diameters, cross-sectional shapes, and spacings between the microneedles. In one embodiment, the microneedles are arranged in a 15 by 15 array with 600 μm tip-to-tip spacing. In one embodiment, the microneedles are arranged in a 20 by 20 array with 600 μm tip-to-tip spacing.

In a further aspect, disclosed herein is a method for treating or preventing thrombosis in a subject in need thereof, comprising:
providing a microneedle patch to a subject, wherein the microneedle patch comprises:
   a plurality of microneedles each having a base end and a tip;
   a substrate to which the base ends of the microneedles are attached or integrated; and
   a hydrogel, wherein the hydrogel comprises:
      a non-peptidic polymer;
      a thrombin-cleavable peptide; and
      heparin;
      wherein the heparin is linked to the non-peptidic polymer by the thrombin-cleavable peptide;
inserting the microneedles into the biological barrier, wherein the heparin is released from the hydrogel upon cleavage of the thrombin-cleavable peptide.

Thrombosis is one of the leading causes of cardiovascular mortalities and morbidities worldwide. Traditionally, routine injections of heparin are utilized to counteract coagulation activation. However, the precise anticoagulant regulation is difficult to achieve by systemic (intravenous) or local (catheter) delivery of anticoagulants. Under- or over-dosage may lead to dangerous consequences due to either rapid clearance in the body or bleeding complications that may lead to spontaneous hemorrhages. The feedback-controlled feature of the thrombin-responsive patch can efficiently avoid the risk of over- or under-dosage and timely deliver the anticoagulant drug. It also provides a painless and convenient administration method. The in vivo studies demonstrate effective protection from acute pulmonary thromboembolism in a long-lasting fashion.

In one embodiment, the stimuli-responsive transcutaneous patch disclosed herein can be applied to specifically deliver anticoagulant and thrombolytic drugs for thrombosis therapy. This system also provides an innovative design guideline for closed-loop based drug delivery systems to treat intravascular diseases according to levels of related biomarkers.

### Examples

The following examples are set forth below to illustrate the compounds, compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Thrombin-Responsive Transcutaneous Patch for Auto-Anticoagulant Regulation

Thrombosis, a pathological hemostatic condition, has become one of the leading causes of cardiovascular mortalities and morbidities worldwide.^{[1,2]} The unwanted intravascular blood thrombi can cause vascular occlusions, organ damage, and severe cardiovascular diseases, including myocardial infarction and stroke.^{[3]} As a first line of defense, anticoagulant drugs can prevent and delay the obstruction in blood flow.^{[2,4]} Heparin (HP), a common anticoagulant, is routinely administered to counteract coagulation activation.^{[5]} Dosing schemes for HP usually involve daily intravenous administration for weeks to months.^{[6]} Unfortunately, systemic (intravenous) or local (catheter) delivery of anticoagulants remains difficult for precise anticoagulant regulation.^{[7]} Under- or over-dosage may lead to dangerous consequences due to either rapid clearance in the body or bleeding complications that may lead to spontaneous hemorrhages.^{[8]} Moreover, it is known that the timely delivery of drugs is critical for cardiovascular patients when an unpredictable attack happens,^{[9]} which makes sustained protection from pathogenesis imperative. Therefore, a controlled and on demand drug delivery system, one that enhances therapeutic efficacy while minimizing side effects and time-to-treatment, is urgently needed for the management of thrombotic diseases.^{[10]}

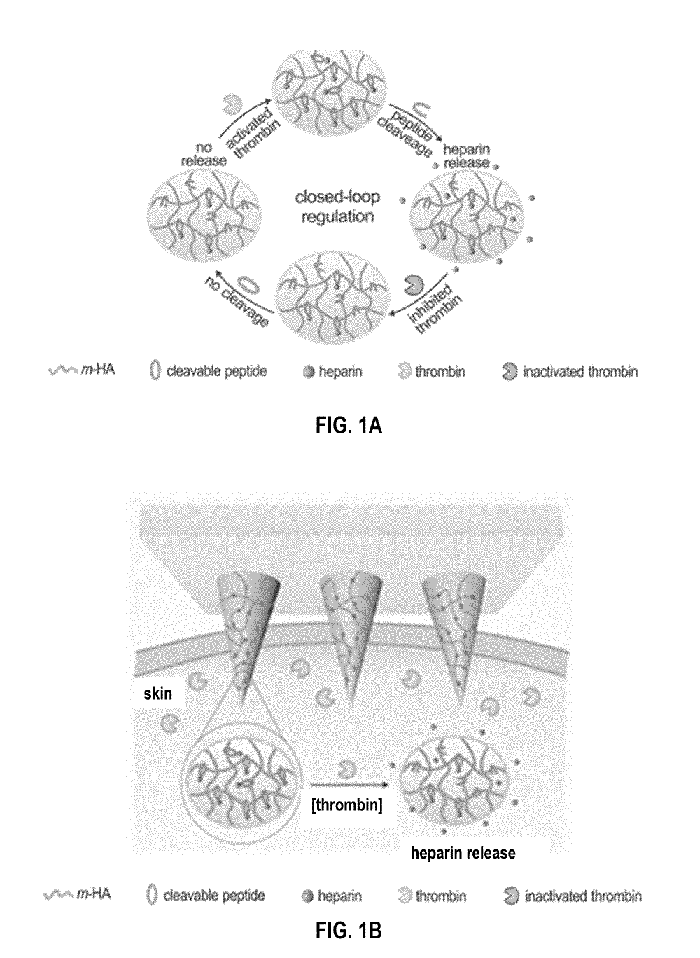

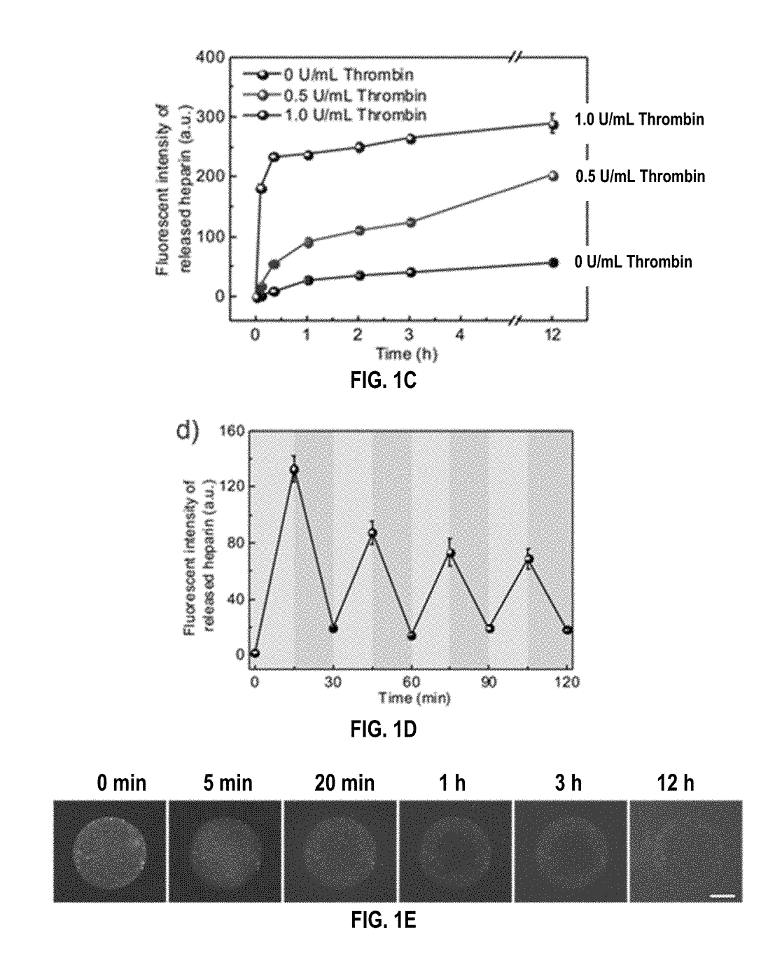

In this example, an engineered feedback-controlled anticoagulant system based on thrombin-responsive polymer-drug conjugates is described. Thrombin is a trypsin-like serine proteinase that plays an imperative role in blood coagulation systems to produce insoluble fibrin from soluble fibrinogen.^{[11]} Recently, thrombin-responsive systems based on the thrombin-cleavable peptide have attracted great attention due to associated high sensitivity and fast response rate.^{[12]} In this system, a thrombin-cleavable peptide is introduced as a linker during the conjugation of HP to the main chain of hyaluronic acid (HA).^{[13]} The peptide can be cleaved when thrombin is activated,^{[14,15]} triggering drug release from the backbone in a thrombin-responsive fashion (FIG. 1a). The thrombin-responsive HP conjugated HA (TR-HAHP) matrix can be obtained via polymerization under ultraviolet (UV) light treatment. In the presence of the elevated thrombin concentration, HP can be promptly released from the TR-HAHP matrix, whereas HP is trapped in the matrix and cannot be released without thrombin. The released HP is able to inhibit the coagulation activation by inactivating thrombin, which suppresses the release of HP from the matrix and minimizes the risk of undesirable spontaneous hemorrhage.

The TR-HAHP derivative can be further integrated into a disposable microneedle (MN) array based transcutaneous device for potential long-term autoregulation of blood coagulation. The micro-size needles on the patch enable convenient administration in a painless manner.^{[16,17]} Owing to the thrombin responsive property, this MN patch acts as a closed-loop “smart” device that can be safely inserted in the skin without drug leaking under a normal blood environment, but rapidly responds to an increased thrombin level and releases a corresponding dose of anticoagulant drug to prevent the undesired formation of blood clots (FIG. 1b). The results show that this “smart” HP patch can offer sustained autoregulation of blood coagulation in a safe and convenient manner.

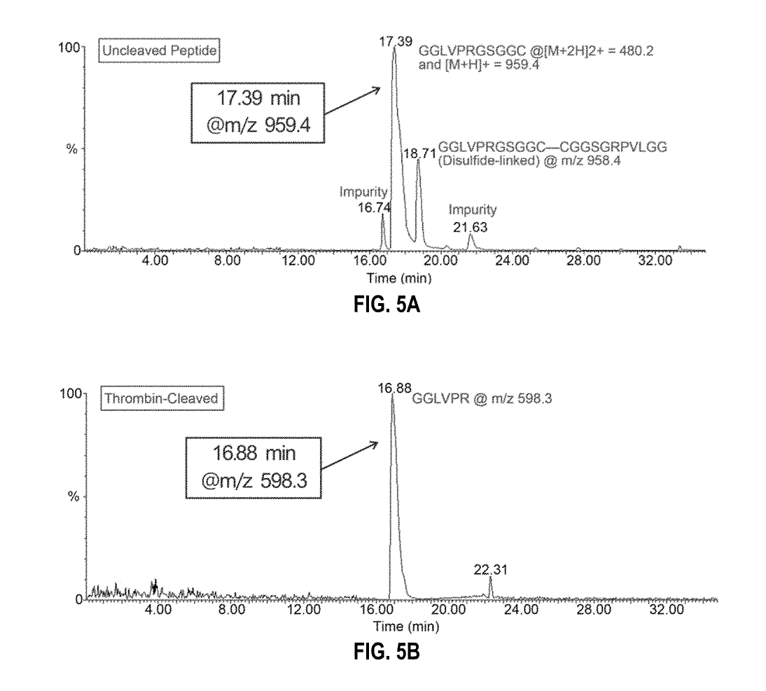

To achieve the stimuli-triggered heparin delivery, a thrombin cleavable peptide with a sequence of GGLVPR|GSGGC (SEQ ID NO:1), was introduced as a linker to obtain the TR-HAHP. The cleavage of the peptide by thrombin was verified by liquid chromatography mass spectrometry (LCMS) analysis, which showed that the peptides were efficiently cleaved after 12 h incubation with 1 U mL⁻¹ thrombin in Tris buffer (20×10⁻³ m Tris, 150×10⁻³ m NaCl, 2.5×10⁻³ m KCl, pH 7.4) (FIG. 5). To prepare the TR-HAHP, the cleavable peptide was first conjugated to the methacrylated HA (m-HA) through the formation of an amide bond. Then, HP was further covalently bound to the cysteine residue of the peptide to obtain the TR-HAHP. In the presence of the activated thrombin, the short peptide can be selectively recognized and cleaved between Arg (R) and Gly (G) to achieve specific HP release.^{[14]} The successful conjugation of HP to m-HA was evidenced by the elemental analysis and the increase in molecular weight from 314 to 606 kDa (Table 1).

TABLE 1
Elemental analysis of m-HA, HP and TR-HAHP.

| Percent (%) | C | O | N | S |
|---|---|---|---|---|
| m-HA | 45.8 ± 0.5 | 35.3 ± 0.4 | 3.8 ± 0.5 | 0.7 ± 0.2 |
| HP | 26.2 ± 0.4 | 39.1 ± 0.5 | 1.4 ± 0.4 | 17.1 ± 0.5 |
| TR-HAHP | 49.7 ± 0.5 | 27.5 ± 0.4 | 7.8 ± 0.5 | 8.6 ± 0.4 |

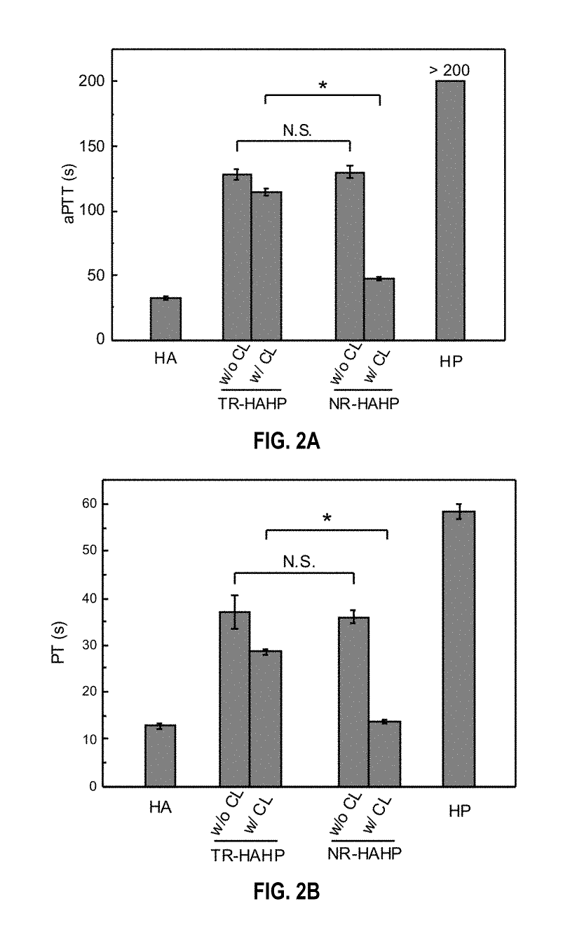

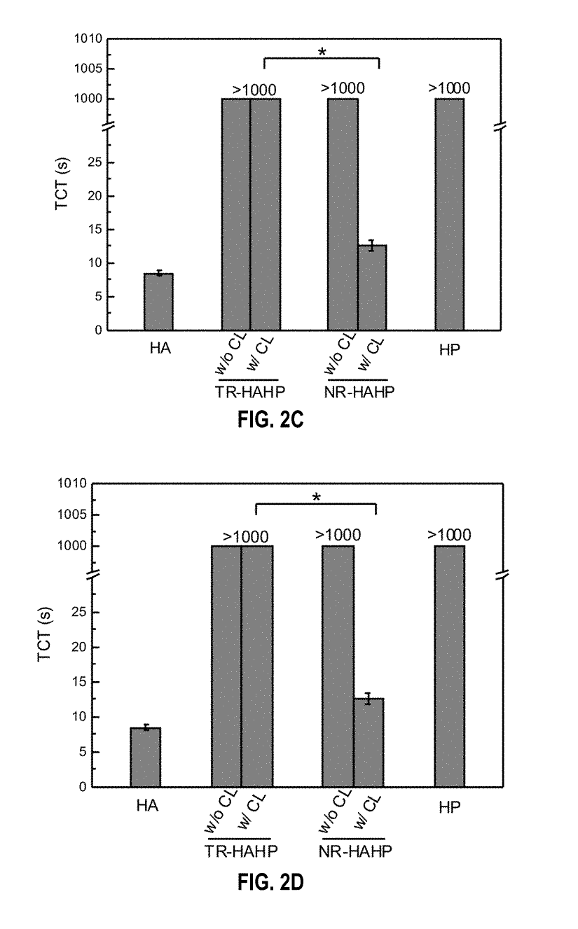

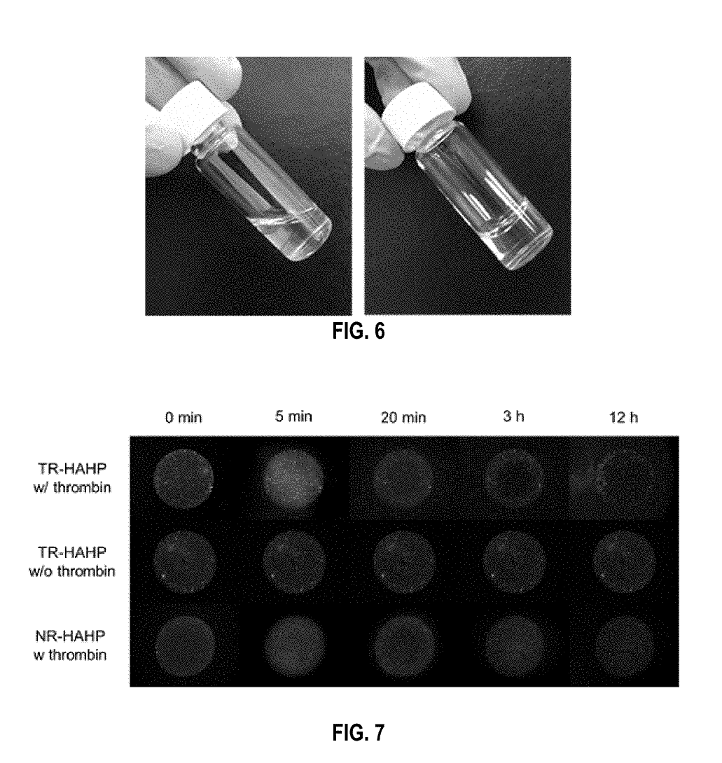

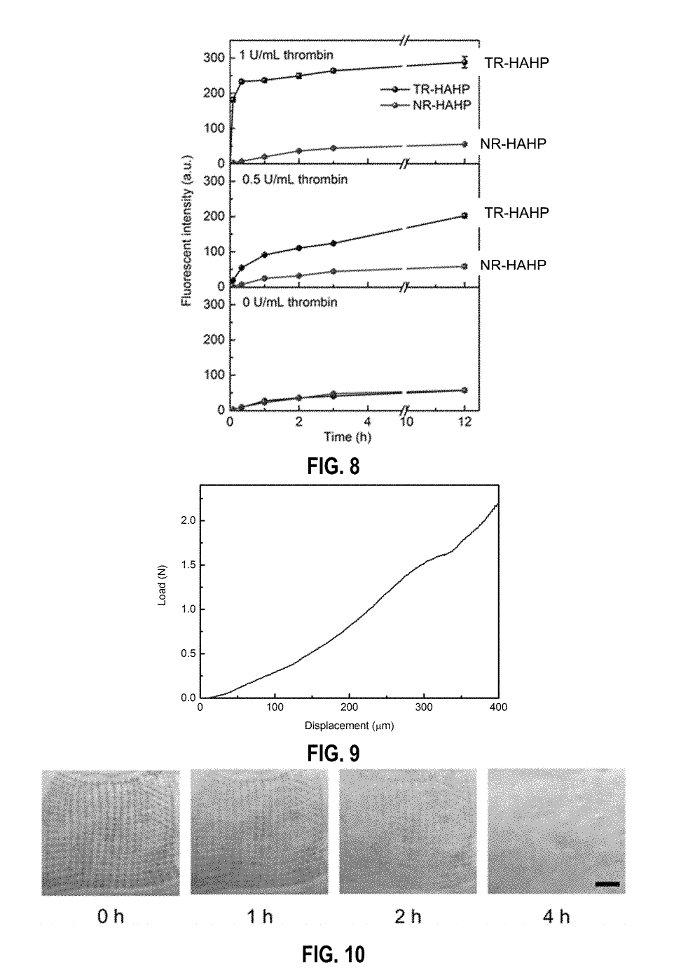

In order to examine the effect of thrombin in the TRHAHP based system, a TR-HAHP hydrogel was prepared via photo-polymerization (FIG. 6). The prepared hydrogels were incubated in thrombin solutions with different concentrations (0, 0.5, and 1 U mL⁻¹), and the release kinetics were obtained by measuring the fluorescence intensity of FITC-labeled HP. As shown in FIG. 2a, the release profiles presented a high dependence on the thrombin level. The TR-HAHP hydrogel quickly responded to the relative higher thrombin concentration (1 U mL⁻¹), and released most of the conjugated HP within 20 min, allowing for fast action of the drug under urgent clinical situations. In contrast, the hydrogel was stable in the buffer without thrombin for up to 12 h (FIG. 1c and FIG. 7). Furthermore, a pulsatile release pattern was observed when the TR-HAHP hydrogel was alternately exposed every 15 min for several cycles to solutions with and without thrombin (FIG. 1d). The hydrogel performed the repeatable and sustained release of HP, corresponding to the presence or absence of thrombin. Additionally, the release process was monitored in real time by fluorescence microscopy. As demonstrated in FIG. 1e, the cleaved FITC-HP gradually diffused through the cross-linked hydrogel after the addition of thrombin, while the hydrogel maintained its original structure during the release period. In contrast, there was insignificant fluorescence signal detected in the buffer without thrombin after 12 h (FIG. 7). To further confirm the thrombin-responsive release, a non-responsive HP-HA conjugate without the thrombin-sensitive peptide (NR-HAHP) was synthesized directly via a heterobifunctional linker as a negative control. From the fluorescence images and release profiles of the NR-HAHP hydrogel incubating with thrombin solutions, it was demonstrated that HP could not detach from the HA matrix without the degradation of the thrombin-sensitive peptide (FIGS. 7 and 8). Collectively, these results suggested that the thrombin-specific activation feature of the TR-HAHP is attributed to the incorporation of the cleavable peptide unit.

To validate the in vitro anticoagulant regulation ability of TR-HAHP, the activated thromboplastin time (aPTT) and prothrombin time (PT) were measured to determine the anticoagulant potency of different samples, including the empty HA hydrogel, HA hydrogel containing free HP, TR-HAHP gel, and NR-HAHP gel by incubation with human plasma. The activated thromboplastin time measurement is commonly used for the evaluation of the intrinsic pathways of blood coagulation,^{[18]} while the PT measurement is a test for the evaluation of extrinsic pathways in clinical medicine.^{[19]} Antithrombin III, a natural thrombin inhibitor, can inactivate thrombin via forming a covalent enzyme complex with thrombin.^{[20]} Since it has a specific heparin binding-site proximal to the pentasaccharide, the inactivation of thrombin by antithrombin III can be promoted by nearly three orders of magnitude in the presence of heparin.^{[21]} As shown in FIG. 2a,b, compared with the healthy human plasma treated with the empty gel, both TRHAHP and NR-HAHP solutions prolonged aPTT and PT by up to 100 s. These prolonged aPTT and PT can be attributed to the existence of heparin based on an antithrombin-dependent mechanism.^{[22]} However, once cross-linked by UV irradiation, the NR-HAHP gel could not inhibit the coagulation while the TR-HAHP gel still showed a remarkable increase in the aPTT and PT levels, indicating the thrombin-specific release of HP from the TR-HAHP gel. The anticoagulant capability of the TR-HAHP was further evaluated via a thrombin clotting time (TCT) assay, which is commonly performed on patients for diagnosis of coagulopathy by adding thrombin to citrated plasma and recording the time when a stable clot is formed.^{[23]} Consistent with the aPTT and PT results, TCT was significantly delayed in the presence of the TR-HAHP gel (FIG. 2c).

The hydrogels were further incubated with human plasma twice with 3 h for each incubation cycle to examine the self-regulation ability of the TRHAHP. Thrombin formation in plasma was determined by the level of the prothrombin F1+2 fragment, which is cleaved from prothrombin during the activation.^{[24]} The coagulation activation levels of the TR-HAHP hydrogel versus non-responsive gels (HP and NR-HAHP) were reported in FIG. 2d. A high level of F1+2 was detected in the plasma incubated with the control groups (HA and NR-HAHP), while both HP gel and the TR-HAHP gel effectively inhibited coagulation activation in the first incubation cycle. In the presence of TR-HAHP hydrogel, plasma was protected from clotting over both investigated periods, whereas plasma in contact with the HP hydrogel could only prevent coagulation in the first incubation cycle due to the burst release of HP from the gel during the incubation. The thrombin responsiveness of the TR-HAHP enabled the controlled and repeatable HP release from the system, as less HP was released once thrombin was inhibited by the pre-released HP. The remarkable difference in F1+2 concentrations between plasmas incubated with the HP gel versus the TR-HAHP gel confirmed that the feedback system could inhibit coagulation over a long time period.

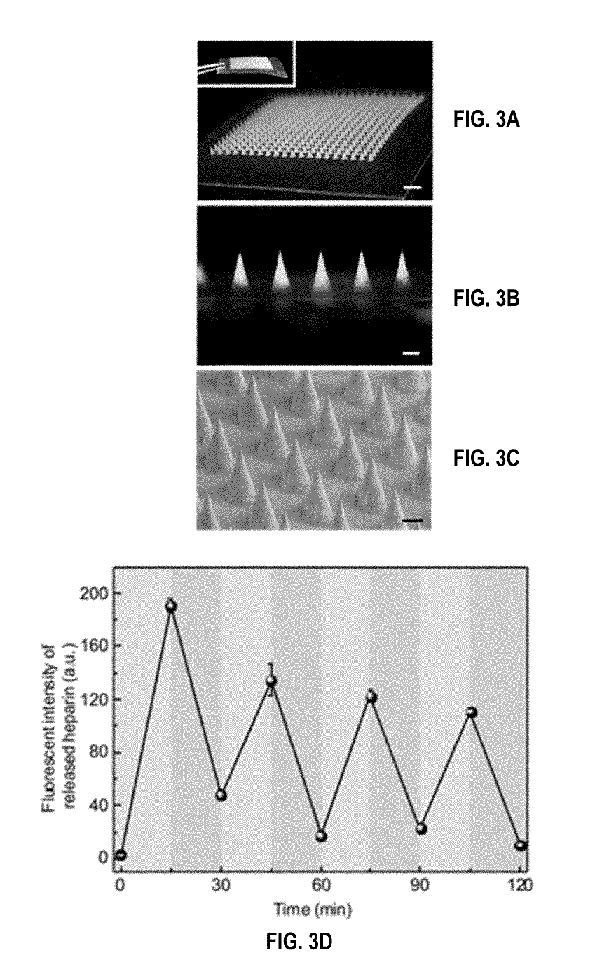

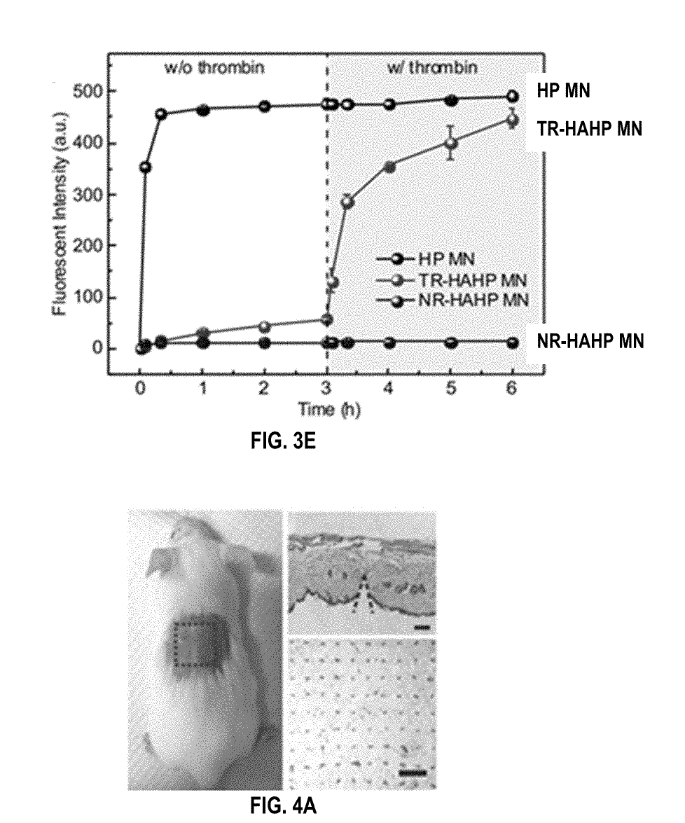

To achieve a functional form that enables painless and convenient HP delivery, a TR-HAHP MN array patch was fabricated to assess long-term anticoagulant regulation. Briefly, the TR-HAHP solution mixed with the cross-linker MBA and a photoinitiator was first loaded into the tip region of a silicone MN mold by centrifugation. The cross-linked HA-based matrix enhances the stiffness of the MNs (FIG. 9) for efficient penetration through the skin,^{[16]} as well as restricts the loss of the TR-HAHP from the MNs. The MN array contains 400 needles in a 12×12 mm2 patch with a 600 μm center-to-center interval (FIG. 3a). Each MN was of a conical shape, with 300 μm in diameter at the base and 600 μm in height (FIG. 3c). The fluorescence image in FIG. 3b displayed a cross-sectional view of the MN with a rhodamine-labeled m-HA matrix and FITC-labeled TR-HAHP loaded in MN tips with a homogenous distribution.

The obtained TR-HAHP MNs exhibited thrombin-responsive performance similar to the TR-HAHP hydrogel. As shown in FIG. 3d, a repeatable release profile of HP was observed corresponding to thrombin levels, which may further enable prolonged thrombin-mediated HP delivery. In addition, tunable release kinetics can be achieved by varying the incubating condition (FIG. 3e). A maximum of a 15.6-fold increase in the HP release rate was observed in 20 min once exposed to thrombin solution (0.6 U mL⁻¹). In contrast, the free HP-loaded MNs exhibited a burst release in the Tris buffer even without thrombin, but an insignificant amount of HP was released from the NR-HAHP MNs.

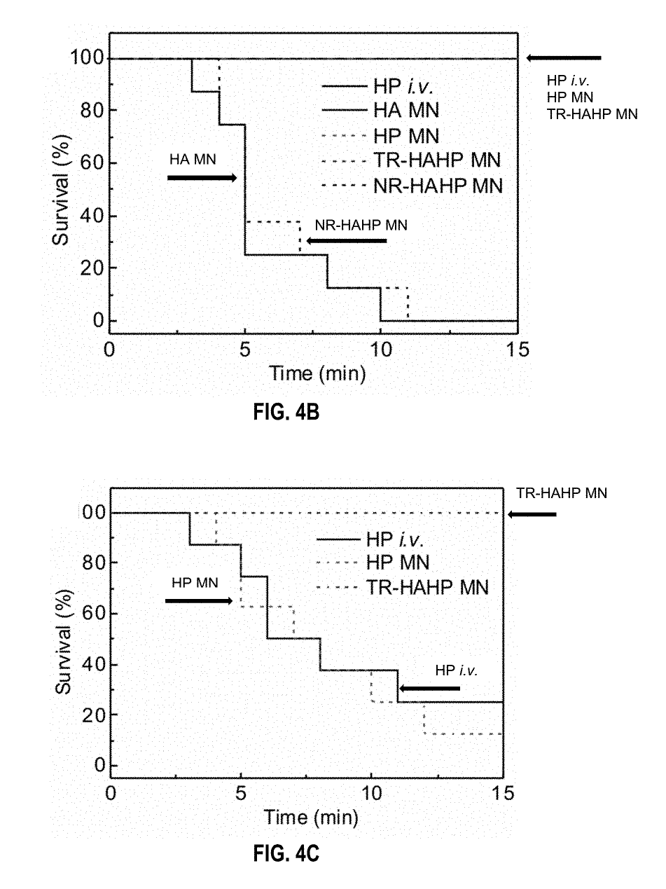

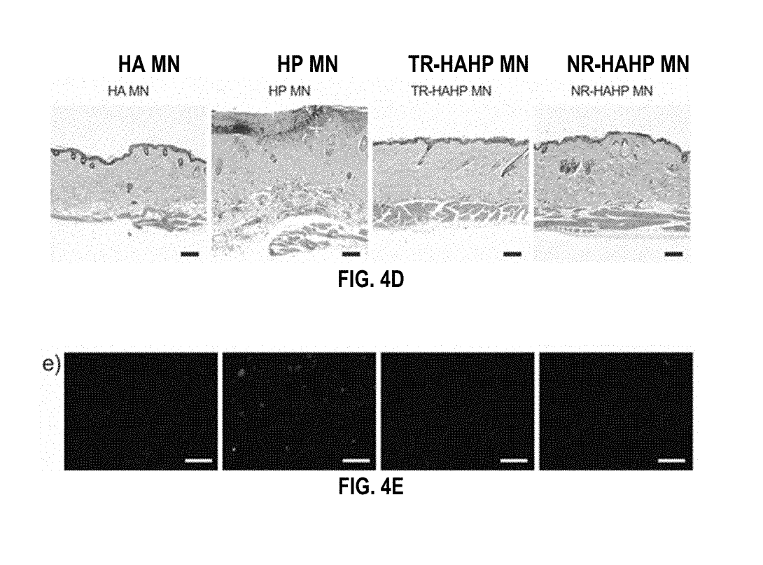

To further evaluate the potential clinical relevance for the treatment of life-threatening acute thrombosis, the anticoagulant capacity of the TR-HAHP was verified in a thrombotic challenge model.^{[25]} The CD-1 mice were randomly divided into five groups (n=8), with one group intravenously (i.v.) injected with heparin solution and four groups transcutaneously administered with different samples: 1) the empty HA MN made of only cross-linked m-HA, 2) the HA MN encapsulating free HP (HP MN), 3) the TR-HAHP MN, and 4) the NRHAHP MN (HP dose: 200 U kg⁻¹). The MNs could penetrate the mouse skin efficiently, as evidenced by the hematoxylin and eosin (H&E) and trypan blue staining of the MN-treated tissue (FIG. 4a), which allowed the MN tips to be exposed to the blood fluid in vascular-capillary network for real-time sensing and rapid response. The transient microchannels in the skin were quickly recovered 4 h post MN injection (FIG. 10).

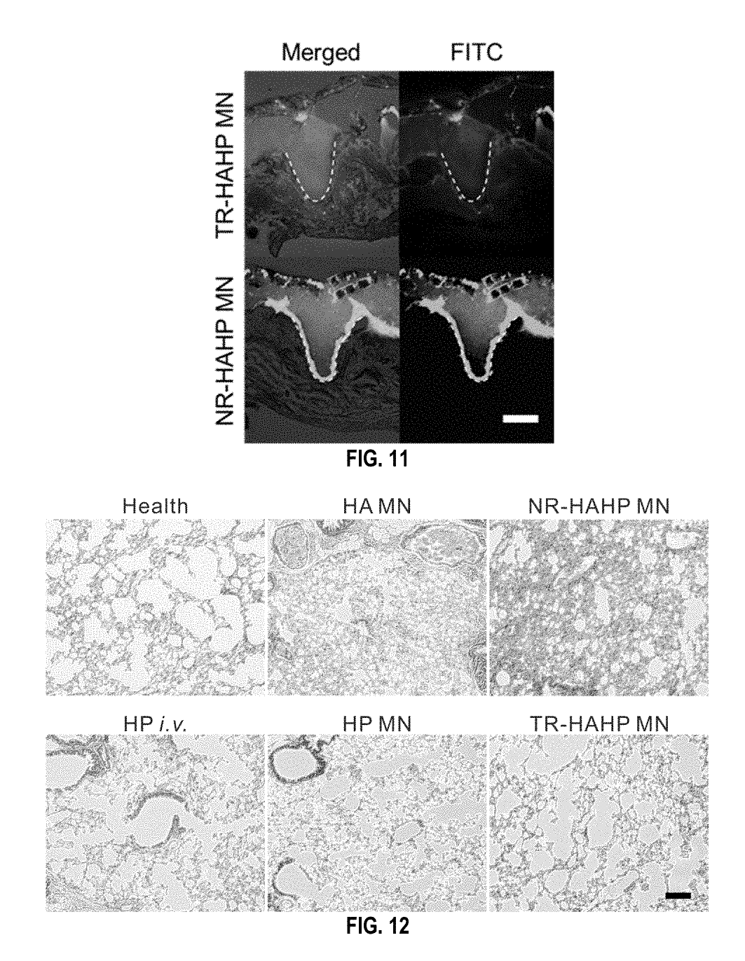

Each mouse was i.v. injected with thrombin (1000 U kg⁻¹) to induce an acute thromboembolism, which can lead to mortality in ≈92% of mice.^{[26]} Heparin solution was i.v. injected into the mice before thrombosis induction. The MN patches were pre-administered on the dorsum skin of the mice 10 min before the challenge to be tested. All animals with empty HA MN or the NR-HAHP MN died within 15 min after the injection of thrombin, whereas all mice survived with the treatment of HP MN or TR-HAHP MN (FIG. 4b) during the 15 min. The significantly enhanced survival rate implied fast and efficient HP release from the TR-HAHP MN in response to increased thrombin, which protected the mice from the thrombolytic risk. Through FITC-labeled heparin, the in vivo release triggered by thrombin was also verified by fluorescence microscopy (FIG. 11). In a further step, the survival rate was also examined 6 h post administration of MN patches and heparin injection. It was demonstrated that ≥80% of mice treated with the HP MNs or i.v. injection of heparin died as a result of the short half-life of HP (≈4 h) (FIG. 4c).^{[27]} The increased mortality rate 6 h post administration of HP MNs suggested that the burst release of HP was not able to ensure protection from thrombotic risk. Contrary to the behavior of HP MN, the TR-HAHP MN maintained its function of anticoagulation and protected the animals from death. The superior anticoagulant capacity of TR-HAHP MN was also evidenced by H&E staining of lung sections. There were insignificant differences observed in the lung of mice treated with TR-HAHP MN compared with healthy mice (FIGS. 12 and 13); but intravascular and interstitial hemorrhage, blocked blood vessels, and atelectasis were observed in the challenged groups 6 h post administration of HP injection or HP MN (FIG. 13). These data indicate that the stimulus-triggered feature of the TR-HAHP system enables its potential application in self-administered therapy.

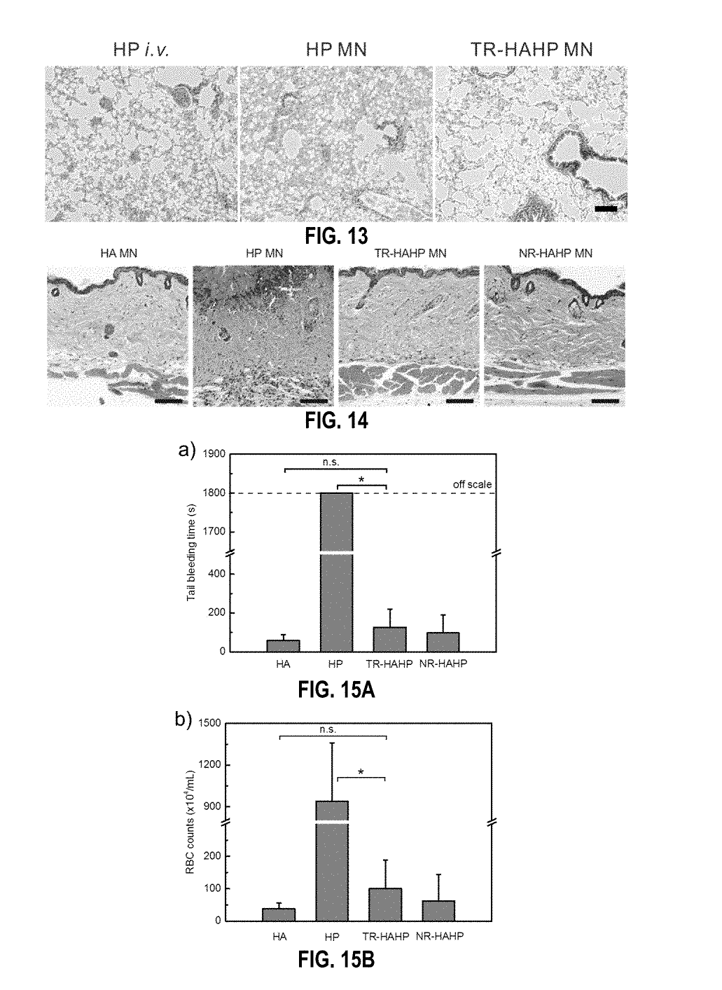

To further investigate the biocompatibility of the MN array patches, the mouse skin surrounding the MN-treated area was excised for histological analysis after 24 h administration. The pure HA MN was regarded as a negative control, which exhibited high biocompatibility as observed in the H&E stained histological images (FIG. 4d and FIG. 14), whereas obvious damage was observed in the skin treated with HP MN. The H&E images indicated neutrophil infiltration and a severe pathophysiological response because the HP caused subcutaneous bleeding. On the contrary, there were insignificant lesions at the TR-HAHP MN treated site because no HP leaked from the MN in the absence of thrombin. Moreover, as presented in the skin tissues stained with the in situ TUNEL assay, obvious cell apoptosis occurred in the skin treated with HP MN, while no significant cell death was observed in the skin treated with the TR-HAHP MN, NRHAHP, and pure HA MN (FIG. 4e). Finally, the TR-HAHP MN avoided unwanted bleeding due to the locally generated, and considerably lower levels of activated thrombin at the sealing of the major wounds,^{[28]} which could not be sensed by the MNs in the treated subcutaneous tissue (FIG. 15).

In conclusion, a thrombin-responsive patch was developed for auto-regulation of blood coagulation by integrating a TRHAHP matrix with a MN-array. The thrombin-cleavable peptide unit enabled thrombin-specific activation of drug release from the system with a rate highly dependent on the thrombin concentration. More importantly, it enabled feedback-controlled anticoagulation therapy with minimized risk of over- or underdosage. The in vivo studies in a thrombolytic challenge model demonstrated effective long-term protection from acute pulmonary thromboembolism. Taken together, this work provides a platform for designing closed-loop based drug delivery systems for the treatment of intravascular diseases according to levels of related biomarkers. Moreover, the integration of MNs with stimuli-responsive drug carriers extends the administration methods of therapeutics.^{[29]}

### Methods

Materials.

All chemicals were purchased from Sigma-Aldrich unless otherwise specified and were used as received. Thrombin cleavable peptide (GGLVPRGSGGC) was ordered from GL Biochem Ltd (Shanghai, China). Heparin with an activity of 212 U/mg was obtained from Sigma-Aldrich. aPTT, PT, TCT reagents and human plasma were purchased from Helena Laboratories, Inc (Beaumont, TA, USA). Human F1+2 ELISA kit was purchased from MyBioSource, Inc (San Diego, Calif., USA). The deionized water was prepared by a Millipore NanoPure purification system (resistivity higher than 18.2 MΩ cm⁻¹).

LC-MS Analysis of Peptide Cleavage.

Peptide with sequence GGLVPR|GSGGC was incubated with thrombin (1 U/mL) in Tris buffer (20 mM Tris, 150 mM NaCl, 2.5 mM KCl, pH 7.4) for 12 h. The LC-MS analysis of intact peptide and cleaved peptide (GGLVPR) was shown in FIG. S1.

Synthesis of Acrylate Modified HA (m-HA).

m-HA was synthesized follow the previously reported method (T. Jiang, R. Mo, A. Bellotti, J. Zhou, Z. Gu, Adv. Funct. Mater. 2014, 24, 2295). Briefly, 2.0 g of HA was dissolved in 100 mL of DI water at 4° C., to which 1.6 mL of methacrylic anhydride (MA) was dropwise added. The reaction solution was adjusted to pH 8-9 by the addition of 5 M NaOH and stir at 4° C. for 24 h. The resulting polymer was obtained by precipitation in acetone, followed by washing with ethanol for 3 times. The product re-dissolved in DI water and the solution dialyzed against DI water for 2 days. m-HA was achieved by lyophilization with a yield of 87.5%. The degree of modification was calculated to be 15% by comparing the ratio of the areas under the proton peaks at 5.74 and 6.17 ppm (methacrylate protons) to the peak at 1.99 ppm (N-acetyl glucosamine of HA) after performing a standard deconvolution algorithm to separate closely spaced peaks. ¹H NMR (300 MHz, D₂O, δ): 1.85-1.96 (m, 3H, CH2=C(CH₃)CO), 1.99 (s, 3H, NHCOCH₃), 5.74 (s, 1H, CH¹H²═C(CH₃)CO), 6.17 (s, 1H, CH¹H²═C(CH₃)CO).

Synthesis of HA-Pep Conjugates.

50 mg of m-HA was mixed with 1-ethyl-3(3-dimethylaminopropyl) carbodiimide (EDC)/N-hydroxysuccinimide (NETS) (117 mg/81 mg) for the activation of carbonyl groups on m-HA in a pH 5.0 sodium acetic buffer for 30 min at RT, and the unreacted EDC and NHS were removed using a centrifugal filter (100,000 Da MWCO, Millipore). Then 30 mg peptide was added to react with m-HA in a pH 7.4 PBS buffer at RT for overnight. Free peptides were removed using a centrifugal filter (100,000 Da MWCO).

Synthesis of TR-HAHP Conjugates.

The carbonyl groups on HP was activated by mixing with EDC/NHS and stirred for 30 min. Then 1,6-diaminohexane was added for another 4 h at RT (pH 8.5). The reaction solution was thoroughly dialyzed against DI water for 1 day and followed by lyophilization (Freeze Dry System, Labconco, Kansas City, Mo., USA) to remove the residual water. The pre-modified HP was mixed with sulfosuccinimidyl-4-(N-maleimidomethyl)-cyclohexane-1-carboxylate (Sulfo-SMCC, Pierce) in PBS (pH 7.4) at a molar ratio of 1:5 for 0.5 h at RT and purified with a centrifugal filter (10,000 Da MWCO). Finally, the activated HP and the HA-Pep conjugates were mixed in PBS (pH 8.0). After 24-h reaction at 4° C., the obtained TR-HAHP was washed with water using a centrifugal filter (100,000 Da MWCO) and stored at 4° C. till use. The elemental analysis of TR-HAHP was measured using a FEI Verios 460L field-emission scanning electron microscope (FESEM) combined with energy dispersive X-ray microanalysis. Fluorescein isothiocyanate (FITC) labelled HP was obtained by mix the FITC with HP for 24 h at RT. The free FITC were removed by a centrifugal filter (10,000 Da MWCO).

Synthesis of NR-HAHP Conjugates.

The NR-HAHP conjugates was prepared by directly mix carbonyl group-activated m-HA with the HP derivative in PBS buffer (pH 7.4) for overnight reaction. Free HP was removed by ultracentrifugation as mention above.

Preparation of TR-HAHP Hydrogel.

Crosslinker N,N′-methylenebisacrylamide (MBA, w/v: 2%) and photoinitiator (Irgacure 2959, w/v: 0.2%) were mixed in TR-HAHP solution. After UV irradiation (wavelength: 365 nm) for 60 s, the mixture underwent the crosslinking polymerization to form the hydrogel.

In Vitro Release Studies.

To evaluate the thrombin-responsive characteristics of TR-HAHP hydrogels, the hydrogels were incubated in Tris buffer (20 mM Tris, 150 mM NaCl, 2.5 mM KCl, pH 7.4) at 37° C. on an orbital shaker, to which various amounts of thrombin were added to reach concentrations at 0, 0.5, and 1 U/mL. At predetermined time points, 100 μL of the supernatant was taken out for analysis by measuring the emission intensity of FITC at 519 nm with the excitation wavelength at 495 nm on the Infinite 200 PRO multimode plate reader (Tecan Group Ltd., Switzerland). To access the hydrogel's ability to adapt to cyclical changes in thrombin concentrations, the TR-HAHP hydrogel was first incubated in Tris buffer with thrombin (0.6 U/mL) for 15 min. At that point, the supernatant was removed and the FITC intensity was measured using the same method mentioned above. Then the hydrogel was incubated in Tris buffer without thrombin for another 15 min. This cycle was repeated numerous times.

The release profiles of FITC-heparin from MNs were monitored by immersing the tips of MNs into Tris buffer with different concentrations of thrombin. At predetermined time points, 100 μL of the medium was taken out, and the fluorescence intensity was then measured using the same method mentioned above to quantify the release amount of FITC-heparin.

Anticoagulant Assays.

In vitro Activated Partial Thromboplastin Time (aPTT) assay and Prothrombin Time (PT) assay were performed to examine the anticoagulant activity of TR-HAHP. Specifically, the hydrogel (HA, HP, NR-HAHP, TR-HAHP), human plasma, and aPTT or PT reagent were mixed together at a ratio of 1:9:10 and incubated at 37° C. for 3 min. Then, 0.025 μM calcium chloride was added to the samples, and the time was recorded for clot formation. For the Thrombin Cloting Time (TCT) assay, the human plasma was first incubated with hydrogel for 3 min at 37° C. Afterwards, the TCT reagent was added into the mixture and the doting time was recorded. The hydrogels with and without crosslink were tested seperately for each assay. To evaluate the thrombin reponsiveness of TR-HAHP, the hydrogels were incubated with human plasma for two cycles. Each cycle was performed at 37° C. under constant revolution and avoiding air contact for 3 h. The blood plasma was removed after the first incubation and replaced with fresh human plasma. After each incubation period, ELISA tests using commercial kits for prothrombin F1+2 fragment was performed.

Fabrication of TR-HAHP MNs.

All the MNs in this study were fabricated using the uniform silicone molds from Blueacre Technology Ltd. Each needle had a 300 μm by 300 μm round base tapering to a height of 600 μm with a tip diameter of around 10 μm. The needles were arranged in a 20×20 array with 600 μm tip-to-tip spacing. To fabricate TR-HAHP MN, TR-HAHP solution with MBA (w/v=2%), photoinitiator (Irgacure 2959, w/v=0.5%) was first deposited by pipet onto the MN mold surface (100 μL/array). Then, molds were placed under vacuum (600 mmHg) for 20 min to allow the solution filled the MN cavities and became more viscose. Afterwards, the covered molds were centrifuged using a Hettich Universal 32R centrifuge for 20 min at 2000 rpm. Finally, 3 mL premixed N,N′-methylenebisacrylamide (MBA, w/v: 2%), photoinitiator (Irgacure 2959, w/v: 0.5%) and m-HA solution(w/v: 4%) was added into the prepared micromold reservior and allowed to dry at 20° C. under vacuum dessicator. After completely desiccation, the MN patch was carefully detached from the silicone mold and underwent the crosslinking polymerization via UV irradiation (wavelength: 365 nm at an intensity of 9 mW/cm²) for 30 s. The resulting MN-array patches were stored in a sealed six well container for later study. The morphology of the MNs was characterized via a FEI Verios 460L field-emission scanning electron microscope.

Mechanical Strength Test.

The mechanical strength of MNs was measured by pressing MNs against a stainless steel plate. The speed of the top stainless steel plate movement towards the MN-array patch was 1 μm/s. The fracture force of MNs was recorded as the needle began to buckle.

Skin Penetration Efficiency Test.

The MN-array was applied to the back of the mouse skin for 30 min. After euthanized by CO₂ asphyxiation, the skin was excited and stained with trypan blue for 30 min for imaging by optical microscopy (Leica EZ4 D stereo microscope).

Biocompatibility Analysis.

To evaluate the biocompatibility of the MN-array patches, mice were euthanized by CO₂ asphyxiation and the surrounding tissues were excised after 24-hour MN administration. The tissues were fixed in 10% formalin for 18 h and then embedded in paraffin, cut into 50 μm sections, and stained using hematoxylin and eosin (H&E) and fluorescent TUNEL staining for histological analysis.

In Vivo Thrombosis Model.

Pulmonary thromboembolism in mice was induced follow the literature (P. Gresele, C. Corona, P. Alberti, G. G. Nenci, Thromb. Haemost. 1990, 64, 80; S. Momi, G. Nenci, P. Gresele, Thromb. Res. 1992, 65, S162). Briefly, female CD-1 mice (Charles Rives, Raleigh, N.C., USA), weighing 20-25 g were used. The animal study protocol was approved by the Institutional Animal Care and Use Committee at North Carolina State University and University of North Carolina at Chapel Hill. Mice were caged and fed a regular diet for at least one week before use. Eight mice for each group were selected and pre-administered with the drugs (HA MN, HAHP MN, NR-HAHP MN, TR-HAHP MN) for tests (HP dose: 5 U/patch). The thrombotic challenge was induced by the rapid i.v. injection of 0.2 mL of bovine thrombin solution (1000 U/kg) into the mouse tail vein. The cumulative end point to be overcome was the immediate death of the animal or prolonged paralysis of the hind limbs (for more than 15 min). The total duration of each experiment was 15 min. The animals which did not die within this time were sacrificed by exposure to CO₂ and will be recorded as survivors. No anesthesia was used during the experiment because of the short duration and because anesthesia has been reported to interfere with thromboembolism in this model (W. Paul, P. Gresele, S. Momi, G. Bianchi, C. Page, Br. J. Pharmacol. 1993, 110, 1565). After sacrifice, the lungs of mice were collected, fixed, and sectioned for H&E staining and observed by optical microscopy.

Tail Bleeding Test.

For safety test of the TR-HAHP MN in vivo, 5 mice (male C57B6, Jackson Lab, U.S.A.) in each group were pretreated with different MN patches (with a dose of 200 U/kg HP) and then placed on a 37° C. heating pad. About 2-4 mm from the tip of the mouse's tail (in about 1 mm diameter), a cut was made with a disposable surgical blade. After transection, the tail was immediately placed in a 50-ml falcon tube filled with 37° C. saline. The bleeding time was recorded up to 30 min, red blood cells were counted in each collected blood sample.

Statistical Analysis.

All results presented are Mean±SD. Statistical analysis was performed using Student's t-test or ANOVA test. With a p value <0.05, the differences between experimental groups and control groups were considered statistically significant.

### REFERENCES CITED IN THIS EXAMPLE

[1] N. Mackman, Nature 2008, 451, 914.
[2] W. H. Geerts, D. Bergqvist, G. F. Pineo, J. A. Heit, C. M. Samama, M. R. Lassen, C. W. Colwell, Chest 2008, 133, 381S.
[3] B. Engelmann, S. Massberg, Nat. Rev. Immunol. 2013, 13, 34; N. Doshi, J. N. Orje, B. Molins, J. W. Smith, S. Mitragotri, Z. M. Ruggeri, Adv. Mater. 2012, 24, 3864; Q. Hu, C. Qian, W. Sun, J. Wang, Z. Chen, H. N. Bomba, H. Xin, Q. Shen, Z. Gu, Adv. Mater. 2016, DOI: 10.1002/adma.201603463.
[4] P. M. Mannucci, M. Franchini, Ann. Med. 2011, 43, 116.
[5] R. Collins, A. Scrimgeour, S. Yusuf, R. Peto, N. Engl. J. Med. 1988, 318, 1162; L. Jin, J. P. Abrahams, R. Skinner, M. Petitou, R. N. Pike, R. W. Carrell, Proc. Natl. Acad. Sci. USA 1997, 94, 14683.
[6] M. Cohen, C. Demers, E. P. Gurfinkel, A. G. Turpie, G. J. Fromell, S. Goodman, A. Langer, R. M. Califf, K. A. Fox, J. Premmereur, N. Engl. J. Med. 1997, 337, 447.
[7] M. Moscucci, J. Invasive Cardiol. 2002, 14.
[8] P. Prandoni, A. W. Lensing, A. Piccioli, E. Bernardi, P. Simioni, B. Girolami, A. Marchiori, P. Sabbion, M. H. Prins, F. Noventa, Blood 2002, 100, 3484.
[9] M. J. Davies, A. Thomas, N. Engl. J. Med. 1984, 310, 1137.
[10] N. Korin, M. Kanapathipillai, B. D. Matthews, M. Crescente, A. Brill, T. Mammoto, K. Ghosh, S. Jurek, S. A. Bencherif, D. Bhatta, Science 2012, 337, 738; C. Chen, S. Li, K. Liu, G. Ma, X. Yan, Small 2016; M. F. Maitz, U. Freudenberg, M. V Tsurkan, M. Fischer, T. Beyrich, C. Werner, Nat. Commun. 2013, 4.
[11] B. Dahlbäck, The Lancet 2000, 355, 1627.
[12] R. Bhat, À. Ribes, N. Mas, E. Aznar, F. Sancenón, M. D. Marcos, J. R. Murguía, A. Venkataraman, R. Martínez-Máñez, Langmuir 2016, 32, 1195; C. Argyo, V. Cauda, H. Engelke, J. Rädler, G. Bein, T. Bein, Chem. Eur. J. 2012, 18, 428.
[13] G. Kogan, L. Šoltés, R. Stern, P. Gemeiner, Biotechnol. Lett 2007, 29, 17.
[14] J. Y CHANG, Eur. J. Biochem. 1985, 151, 217.
[15] R. J. Jenny, K. G. Mann, R. L. Lundblad, Protein Expr. Purif. 2003, 31, 1.
[16] M. R. Prausnitz, R. Langer, Nat. Biotechnol. 2008, 26, 1261.
[17] J. Yu, Y Zhang, Y Ye, R. DiSanto, W. Sun, D. Ranson, F. S. Ligler, J. B. Buse, Z. Gu, Proc. Natl. Acad. Sci. USA 2015, 112, 8260; S. P. Sullivan, D. G. Koutsonanos, M. del Pilar Martin, J. W. Lee, V. Zarnitsyn, S.-O. Choi, N. Murthy, R. W. Compans, I. Skountzou, M. R. Prausnitz, Nat. Med. 2010, 16, 915; P. C. DeMuth, Y. Min, B. Huang, J. A. Kramer, A. D. Miller, D. H. Barouch, P. T. Hammond, D. J. Irvine, Nat. Mater. 2013, 12, 367; R. F. Donnelly, T. R. R. Singh, M. J. Garland, K. Migalska, R. Majithiya, C. M. McCrudden, P. L. Kole, T. M. T. Mahmood, H. O. McCarthy, A. D. Woolfson, Adv. Funct. Mater. 2012, 22, 4879.
[18] D. Basu, A. Gallus, J. Hirsh, J. Cade, N. Engl. J. Med. 1972, 287, 324; J. W. Eikelboom, J. Hirsh, Thromb. Haemost. 2006, 96, 547.
[19] A. J. Quick, J. Biol. Chem. 1935, 109, xxiii.
[20] R. D. Rosenberg, N. Engl. J. Med. 1975, 292, 146; P. S. Damus, M. Hicks, R. D. Rosenberg, Nature 1973, 246, 355.
[21] J. P. Sheehan, J. E. Sadler, Proc. Natl. Acad. Sci. USA 1994, 91, 5518; J. Hirsh, N. Engl. J. Med. 1991, 324, 1565.
[22] R. D. Rosenberg, Am. J. Med. 1989, 87, S2.
[23] V. Ignjatovic, Haemostasis: Methods and Protocols 2013, 131.
[24] M. Boisclair, D. Lane, H. Philippou, S. Sheikh, B. Hunt, Thromb. Haemost. 1993, 70, 253.
[25] S. Momi, G. Nenci, P. Gresele, Thromb. Res. 1992, 65, S162.
[26] S. Momi, M. Nasimi, M. Colucci, G. G. Nenci, P. Gresele, Haematologica 2001, 86, 297.
[27] J. Hirsh, W. Van Aken, A. Gallus, C. Dollery, J. Cade, W. Yung, Circulation 1976, 53, 691.
[28] K. E. Brummel, S. G. Paradis, S. Butenas, K. G. Mann, Blood 2002, 100, 148; M. Comer, K. Cackett, S. Gladwell, L. Wood, K. Dawson, J. Thromb. Haemost. 2005, 3, 146.
[29] Y Lu, A. A. Aimetti, R. Langer, Z. Gu, Nat. Rev. Mater. 2016, 1, 16075.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A hydrogel comprising:
a non-peptidic polymer;
a thrombin-cleavable peptide; and
heparin;
wherein the heparin is linked to the non-peptidic polymer by the thrombin-cleavable peptide.

2. The hydrogel of claim 1, wherein the non-peptidic polymer is hyaluronic acid.

3. The hydrogel of claim 1, wherein the non-peptidic polymer is methacrylated hyaluronic acid.

4. The hydrogel of claim 1, wherein the thrombin-cleavable peptide is about 5 to about 30 amino acids in length.

5. The hydrogel of claim 1, wherein the thrombin-cleavable peptide comprises a sequence of GGLVPRGSGGC (SEQ ID NO:1).

6. The hydrogel of claim 1, wherein the heparin is unfractionated heparin.

7. A device for transport of a material across a biological barrier of a subject comprising:
a plurality of microneedles each having a base end and a tip;
a substrate to which the base ends of the microneedles are attached or integrated; and
a hydrogel, wherein the hydrogel comprises:
a non-peptidic polymer;
a thrombin-cleavable peptide; and
heparin;
wherein the heparin is linked to the non-peptidic polymer by the thrombin-cleavable peptide.

8. The device of claim 7, wherein the non-peptidic polymer is hyaluronic acid.

9. The device of claim 7, wherein the non-peptidic polymer is methacrylated hyaluronic acid.

10. The device of claim 7, wherein the thrombin-cleavable peptide is about 5 to about 30 amino acids in length.

11. The device of claim 7, wherein the thrombin-cleavable peptide comprises a sequence of GGLVPRGSGGC (SEQ ID NO:1).

12. The device of claim 7, wherein the heparin is unfractionated heparin.

13. A method for treating or preventing thrombosis in a subject in need thereof, comprising:
providing a microneedle patch to a subject, wherein the microneedle patch comprises:
a plurality of microneedles each having a base end and a tip;
a substrate to which the base ends of the microneedles are attached or integrated; and
a hydrogel, wherein the hydrogel comprises:
a non-peptidic polymer;
a thrombin-cleavable peptide; and
heparin;
wherein the heparin is linked to the non-peptidic polymer by the thrombin-cleavable peptide;
inserting the microneedles into the biological barrier, wherein the heparin is released from the hydrogel upon cleavage of the thrombin-cleavable peptide.

14. The method of claim 13, wherein the non-peptidic polymer is hyaluronic acid.

15. The method of claim 13, wherein the non-peptidic polymer is methacrylated hyaluronic acid.

16. The method of claim 13, wherein the thrombin-cleavable peptide is about 5 to about 30 amino acids in length.

17. The method of claim 13, wherein the thrombin-cleavable peptide comprises a sequence of GGLVPRGSGGC (SEQ ID NO:1).

18. The method of claim 13, wherein the heparin is unfractionated heparin.
